(19) United States
(12) Patent Application Publication
(10) Pub. No.: US 2025/0249215 A1
(43) Pub. Date: Aug. 7, 2025

(54) STEERABLE SHEATH

(71) Applicant: Centerline Biomedical, Inc., CLEVELAND, OH (US)

(72) Inventors: Greg GABAY, CLEVELAND, OH (US); Vikash GOEL, CLEVELAND, OH (US); Hannah HOFACKER, CLEVELAND, OH (US); Brian WENZEL, CLEVELAND, OH (US); Sonya SNYDER, CLEVELAND, OH (US)

(21) Appl. No.: 19/042,866

(22) Filed: Jan. 31, 2025

Related U.S. Application Data
(60) Provisional application No. 63/548,620, filed on Feb. 1, 2024.

(51) Int. Cl. A61M 25/06

(52) U.S. Cl. CPC A61M 25/0662

(57) ABSTRACT

In a described example, a sheath has an elongate tubular sidewall that includes a deflectable distal body end portion that extends from a proximal body end portion. The distal body end portion includes a deflection control portion extending axially along part of the tubular sidewall of the distal body end portion, such as defining a cylindrical sector of the tubular sidewall. The deflection control portion has a different stiffness relative to at least a diametrically opposed axially extending sector of the tubular sidewall of the distal body end portion to provide a deflection zone of the distal body end portion multiple different bend diameters.

### CROSS-REFERENCE TO RELATED APPLICATION

This application claims the benefit of priority to U.S. Provisional Patent App. No. 63/548,620, filed Feb. 1, 2024, which is incorporated herein by reference in its entirety.

### TECHNICAL FIELD

This disclosure relates to a steerable sheath.

### BACKGROUND

A sheath (also referred to as an introducer or guiding sheath) can be used to introduce catheters or other devices (e.g., therapeutic or diagnostic devices) into vascular and non-vascular locations for interventions within a patient's body. In some applications, a guidewire is introduced into the body and the sheath is advanced over the guidewire to position the sheath at or near a desired site. In other examples, the sheath is introduced into the body without a guidewire. Other objects and devices can be inserted into the body through the sheath.

### SUMMARY

This description relates to a steerable sheath having multiple bend diameters (also referred to herein as a bend radius or bend angle or curvature).

As one example, a sheath includes an elongate tubular body having a cylindrical sidewall defining a lumen and having longitudinally spaced apart proximal and distal body end portions. The distal body end portion includes a first sector segment extending longitudinally along the distal body end portion between first and second ends thereof. The first sector segment is adapted to provide the sidewall of the distal body end portion along the first sector segment a different stiffness than a diametrically opposed portion of the sidewall of the distal body end portion. The distal body end portion is configured for bidirectional deflection thereof with different amounts of curvature in opposing first and second directions along a virtual plane extending through the first sector segment and a central axis of the distal body end portion.

As another example, an elongate sheath includes a deflection varying portion extending longitudinally within a sidewall of a deflectable distal body end portion. The deflection varying portion has a relative stiffness that differs from at least a diametrically opposed axially portion of the sidewall of the distal body end portion. A portion of the distal body end portion extends distally from the deflection varying portion. The distal body end portion has different bend diameters for deflection along a plane in a first direction towards the deflection varying portion and/or in a second direction away from the deflection varying portion depending on the relative stiffness.

As yet another example, a sheath includes an elongate tubular body having a cylindrical sidewall defining a lumen and having longitudinally spaced apart proximal and distal body end portions. The distal body end portion includes a first semi-cylindrical sector segment defining a deflection control region extending longitudinally along the distal body end portion between first and second ends thereof. The deflection control region has a different stiffness relative to a diametrically opposed region of the distal body end portion, such that the distal body end portion is configured to provide for bidirectional deflection thereof with different amounts of curvature in opposing first and second directions along a virtual plane extending through the deflection control region, the diametrically opposed region of the distal body end portion, and a central axis of the distal body end portion.

### BRIEF DESCRIPTION OF THE DRAWINGS

FIGS. 1 and 1A are diagrams depicting an example sheath device having more than one bend diameter.

FIG. 2 is a side view of an example sheath.

FIG. 3 is an enlarged view of part of the sheath of FIG. 2.

FIG. 4 is a partial exploded view of the sheath of FIG. 3.

FIG. 5 is a hidden view of the sheath of FIG. 3 showing additional features within the sheath hidden in the view of FIG. 3.

FIG. 6 is an enlarged view of an intermediate part of the sheath of FIG. 5.

FIG. 7 is a cross-sectional view taken along line 7-7 of FIG. 2.

FIG. 8 is a cross-sectional view taken along line 8-8 of FIG. 2.

FIG. 9 is a cross-sectional view taken along line 9-9 of FIG. 2.

FIG. 10 is a cross-sectional view taken along line 10-10 of FIG. 2.

FIG. 11 is a cross-sectional view taken along line 11-11 of FIG. 2.

FIG. 12 is an enlarged view of a distal end part of the sheath of FIG. 5.

FIG. 13 is a cross-sectional view taken along line 13-13 of FIG. 12.

FIGS. 14 and 15 depict another example of a sheath having variable deflection.

### DETAILED DESCRIPTION

This description relates to a steerable sheath having multiple and/or variable bend diameters.

For example, a sheath has an elongate tubular sidewall that includes a deflectable distal body end portion that extends from a proximal body end portion. The distal body end portion includes a deflection control portion extending axially along part of the tubular sidewall of the distal body end portion, such as defining a cylindrical sector of the tubular sidewall. The deflection control portion has a different stiffness relative to at least a diametrically opposed axially extending sector of the tubular sidewall of the distal body end portion. A portion of the tubular sidewall can extend distally from the deflection varying sector to terminate in an open distal end of the sheath. The deflection control portion provides the distal body end portion different bend diameters (e.g., different amounts of curvature) for deflection in opposite directions along a virtual plane that extends through both a longitudinal axis of the distal body end portion and the deflection control portion.

The bend diameters can be defined according to the relative stiffness of the deflection control portion and the other parts of the tubular sidewall in the distal body end portion. The relative stiffness can result from the mechanical properties of the materials used to form the deflection control portion and other parts of the sheath and/or depend on the sidewall thickness of the deflection control portion and such other parts of the distal body end portion. In an example, the tubular sidewall, including the deflection control portion thereof, are polymer materials, which can be extruded or formed by other methods. The edges of the deflection varying sector and other parts of the tubular sidewall of the distal body end portion can be joined together (e.g., by fusing end to end and along edges thereof) to form a monolithic tubular sidewall for the distal body end portion.

Additionally, the sheath can be steerable by using one or more actuation mechanisms, which can include the use of steering cables, magnetic actuation, material driven means (e.g., shape memory materials and/or conducting polymers), and hybrid actuation mechanisms that include two or more of the above actuation mechanisms. In some examples, the sheath includes an anchor ring near the distal end of the distal body end portion and steering cables (e.g., pull wires or tendons) coupled to the anchor ring at equally spaced apart (e.g., diametrically opposed) locations and extending longitudinally through the sheath to a manipulator (e.g., actuator), such as located in a handle from which the sheath extends. The manipulator can be a lever, a dial or other mechanism to move the steering cables within the sheath for moving the steering cable through the sheath for deflecting the distal end body portion.

In a further example, to limit (e.g., constrain) deflection in a given plane, the deflection control portion has a semi-cylindrical cross-sectional shape forming half of the circumference of the deflection zone. This effectively limits the deflection in the given plane resulting in a smaller bend diameter.

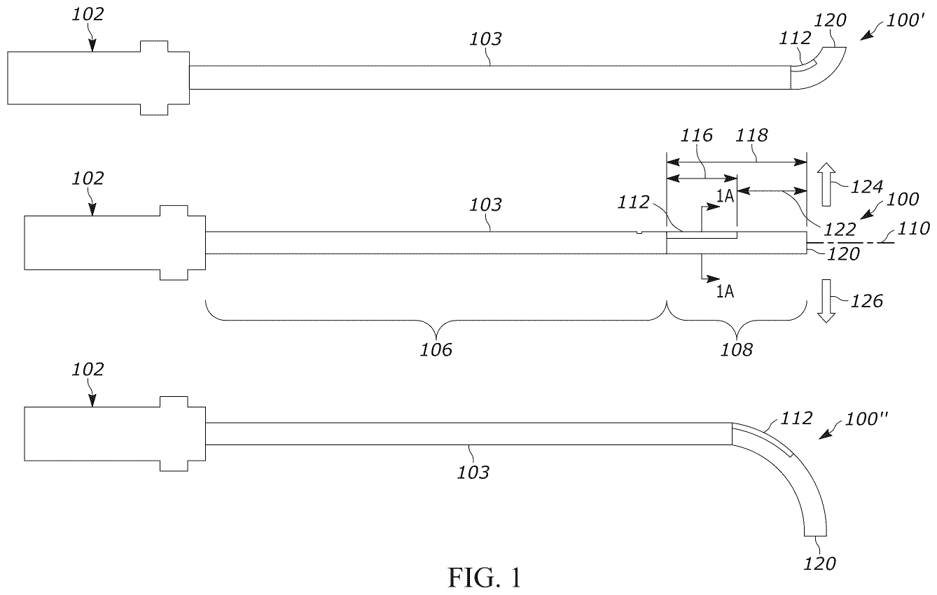

FIG. 1 depicts an example steerable sheath 100 extending from a handle 102. The sheath 100 includes an elongate tubular body (also referred to as a sidewall) 103 defining a main lumen 104 extending through the sheath (see, e.g., cross-sectional view of FIG. 1A). For example, the elongate tubular body has a right-circular cylindrical configuration. Though other tubular shapes can be used to implement the sheath 100 in other examples. The sheath 100 includes longitudinally spaced proximal and distal body end portions 106 and 108, respectively. The distal body end portion 108 of the sheath 100 defines a deflection zone of the sheath 100, which is adapted to provide different bend diameters thereof, as described herein. As used herein, the bend diameter refers to an amount of curvature that the distal end body portion exhibits in response to application of steering force. The steering force can be applied within the sheath (e.g., by one or more steering wires) and/or through an external force (e.g., magnetic steering).

In an example, the distal end body portion 108 is configured to provide different bend diameters in opposite directions along a virtual plane that extends through both a longitudinal axis 110 of the distal body end portion and a deflection control portion 112 (e.g., the virtual plane is coplanar with or parallel to the page on which FIG. 1 is presented). The deflection control portion 112 has a stiffness that is different from at least a diametrically opposed portion of the distal end body portion 108. In one example, the deflection control portion 112 has a stiffness that is greater than other portions of the distal end body portion 108. In another example, the deflection control portion 112 has a stiffness that is less than other portions of the distal end body portion 108. The stiffness of the deflection control portion can be substantially constant along its longitudinal and circumferential directions or it can vary along its longitudinal and/or circumferential directions.

As used herein, stiffness refers to an extent to which a designated portion of the sheath resists deformation in response to an applied force. In some examples, stiffness can be measured according to a durometer scale (e.g., a Shore durometer of the materials). Other measures of stiffness can also be used. Alternatively, or additionally, a measure of the sheath's compliance (e.g., how much the sheath moves or bends in response to an applied force) can be used to quantify the mechanical properties of the sheath. The bending of the distal end body portion 108 in each of its respective deflections can be elastic deformation responsive to the applied steering force and, after the steering force is removed or another force is applied, the material can return to or approximate its original shape.

As shown in the example of FIGS. 1 and 1A, distal body end portion 108 has a circular cylindrical cross-sectional shape and the deflection control portion 112 defines a longitudinally extending sector segment of the cylindrical sidewall 103 of the distal body end portion 108. The deflection control portion 112 thus has a cross-sectional arc length 114 that is less than or equal to about one-half the circumference of the sidewall 103 of the distal body end portion 108. In one example, the longitudinally extending sector segment of the deflection control portion 112 has a semi-cylindrical cross-sectional shape. The longitudinally extending sector segment of the deflection control portion 112 also has an axial length 116, which can also be less than an axial length 118 of the distal body end portion 108. The arc length 114 of the deflection control portion 108, which is defined by the angle θ, and the axial length 116 can be defined (e.g., as design parameters of the sheath 100) to establish the different bend angles.

The distal body end portion 108 extends longitudinally from the proximal body end portion 106 to terminate in distal end 120 of the sheath 100. The longitudinally extending sector segment that defines the deflection control portion 112 also has proximal and distal ends spaced apart from each other by edges that extend the axial length longitudinally along the sidewall of the distal body end portion. The edges of the sector segment are spaced apart from each other to define the arc length 114 of the deflection control portion 112. In the example shown, the arc length 114 at the proximal and distal ends of the deflection control portion 112 can be equal. In other examples, the arc length at each of the proximal and distal ends of the deflection control portion 112 can be different, which can affect the amount of bending along the length thereof. The distal end 120 of the sheath 100 can be spaced axially apart from the distal end of the deflection control portion 112 by a distance shown at 122.

The different relative stiffness of the deflection control portion 112 and the rest of the distal body end portion 108 and the dimensions of the sector segment (e.g., axial length 116 and arc length 114) collectively define the different bend angles for the distal end body portion 108. The bend directions are shown by arrows 124 and 126, and different bend angles are shown for the sheath at 100′ and 100″. In the example of FIG. 1, where the deflection control portion 112 is configured to limit deflection (e.g., has a stiffness greater than the diametrically opposed portion of the distal body end portion 108), the bend angle of the sheath 100′ is less than the bend angle 100″. In an example where the deflection control portion 112 is configured to increase deflection (e.g., has a stiffness less than the diametrically opposed portion of the distal body end portion 108), the bend angle of the sheath 100′ would be less than the bend angle 100″. As described herein, the different bend angles 100′ and 100″ can result from using different materials in each the deflection control portion 112 and the diametrically opposed portion of the distal body end portion 108. Also, or as an alternative, the different bend angles 100′ and 100″ can result from using adding materials to (e.g., adding one or more layers to) and/or removing materials from (e.g., cutting, laser cutting, etching, drilling, and the like partially or wholly through the deflection control portion 112. Also, or as an alternative, portion of the distal body end portion 108 opposite of the deflection control portion 112 can also be modified (e.g., by adding or removing materials) therefrom to result in a different stiffness than the deflection control portion 112.

As an example, to deflect the distal end body portion along a deflection zone, which includes the deflection control portion 112, an anchor can be coupled to (e.g., formed in or otherwise attached to the sidewall 103 of the distal body end portion 108 at a location between a distal end of the sector segment 112 and the distal end 120, such as near the distal end 120. Respective steering cables (e.g., wires or tendons—not shown) can be coupled to the anchor to provide for the bidirectional deflection of the distal body end portion 108 in the first and second directions 124 and 126, such as shown at 100′ and 100″. Opposite ends of the steering cables can be coupled to a manipulator implemented at the handle 102. Various types of manipulators (e.g., knobs, dials, levers, and the like) can be used in the handle 102 to enable deflection of the distal end portion. The handle 102 (or another portion of the sheath) can include markings (e.g., arrows) to specify the steering directions, including an indication of which steering direction has the smaller and larger bend diameters.

As a further example, the distal body end portion 108 of the sidewall 103 is made of one or more polymers. The deflection control portion can be made of a similar polymer material having a different stiffness than the remaining part of the distal body end portion 108. In an example, edges and respective ends of the sector segment 112 abut and are joined with the sidewall 103 of the distal body end portion 108, such as by fusing adjacent edges) to form a monolithic tubular sidewall of the distal end body portion 108. In other examples, the deflection control portion 112 of the distal end portion can have its different stiffness and be formed integrally with the rest of the sidewall 103 of the distal body end portion 108, such as through an extrusion or other fabrication process (e.g., 3D printing or injection molding). Also, or as an alternative, the distal body end portion 108, including the deflection control portion 112 thereof, can be formed of other materials (e.g., nitinol, stainless steel, or other suitable material) or any combination materials to establish a desired stiffness and provide desired bend angles for the distal body end portion 108.

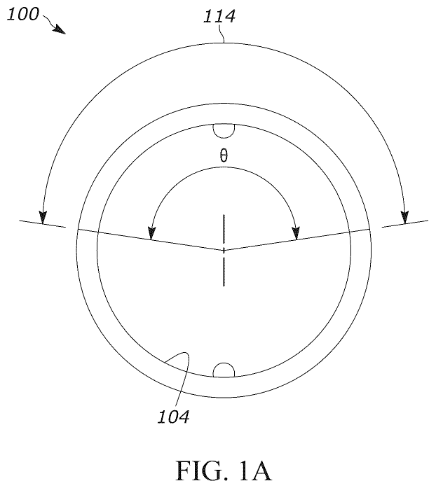

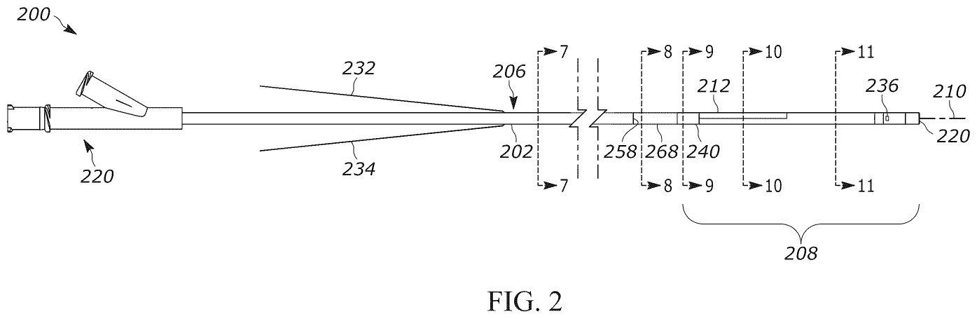

FIGS. 2-13 depict various views of an example of a bidirectional steerable sheath 200, such as can be used to implement the sheath 100 of FIG. 1. Accordingly, the description of FIGS. 2-13 can also refer to certain aspects of the example sheath 100 of FIG. 1. FIG. 2 depicts a side view of the sheath 200 in which a handle has been omitted. Various handles could be used in conjunction with the sheath, such as the handle 102, or otherwise.

The sheath 200 includes an elongate tubular body (also referred to as a sidewall) 202 having longitudinally spaced proximal and distal body end portions 206 and 208, respectively. The sheath 200 has a longitudinal axis 210 extending through the sheath, and the distal body end portion 208 defines a deflection zone of the sheath 200 that is moveable relative to the axis 210. The distal body end portion 208 also includes a deflection control portion 212 having a different stiffness to provide the distal body end portion with different bend angles along a plane extending through the deflection control portion 212 and parallel with the axis.

In the example of FIG. 2, the sheath 200 also includes one or more ports 230 at a proximal end of the proximal end portion 206. The one or more ports 230 are in fluid communication with an interior lumen of the tubular body 202, which can be used to introduce other medical devices (e.g., a guidewire, probe or the like) through the sheath and into the body.

The sheath 200 also includes steering cables (e.g., pull wires, filaments, or tendons) 232 and 234. The steering cables 232 and 234 can be metal or polymer materials. The steering cables 232 and 234 have distal ends coupled to an anchor ring 236 near a distal end 220 of the distal body end portion 208, such as coupled to equally spaced apart (e.g., diametrically opposed) locations of the anchor ring 236 to define respective steering directions. While the sheath 200 in FIG. 2 includes a pair of steering cables, there can be any number of two or more steering cables to provide multi-directional steering. The steering cables 232 and 234 can traverse the tubular body 202 through a respective lumen, and exit through apertures in the tubular body to terminate in proximal ends. The proximal ends of the steering cables 232 and 234 can be coupled to a manipulator (e.g., actuator), such as located in a handle (not shown). The sheath 200 can include one central lumen through which the steering cables 232 and 234 traverse or an individual separate lumen can be used for each respective steering cable.

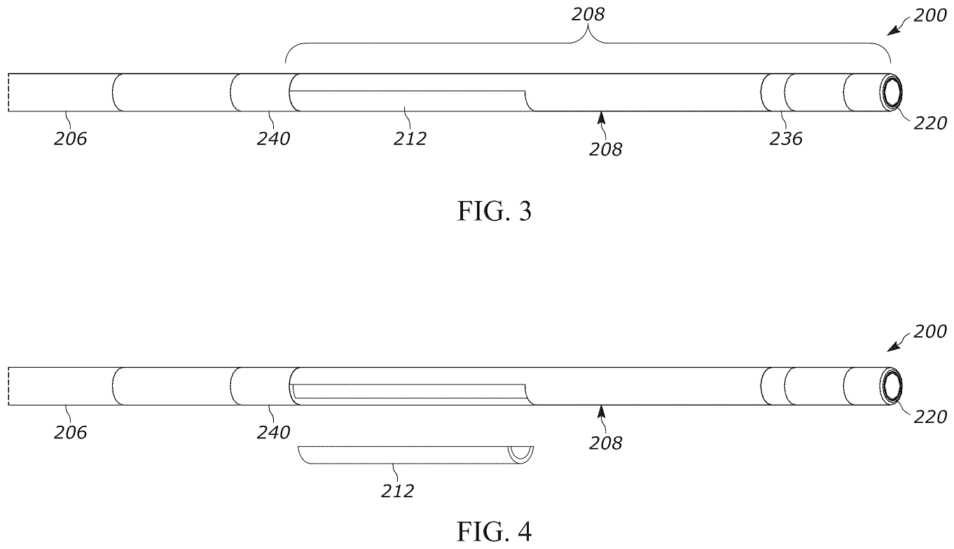

FIG. 3 is an enlarged view of part of the sheath 100 of FIG. 2, and FIG. 4 is a partial assembly view of the sheath of FIG. 3, in which the deflection control portion (e.g., a cylindrical sector) 212 has been exploded from the distal body end portion 208. In the example shown in FIG. 3, the deflection control portion 212 includes an elongated sector segment is separately formed and joined to the distal body end portion 208, such as by fusing (e.g., by application of heat or ultrasonic welding) peripheral edges of the sector segment with corresponding edges of the aperture in the distal body end portion where a corresponding portion thereof has been removed.

There can be one or more transition regions between the proximal and distal body portions 206 and 208. As shown in FIG. 4, a transition region 240 of the tubular body 202 is located between a proximal end of the sector segment 212 and the proximal body end portion 206. The transition region 240 can be formed of a material having a stiffness that is less than the remaining proximal body end portion 206 and equal to or greater than the cylindrical sector segment 212 being attached. Also, the cylindrical sector segment 212 can define a deflection limiter having a stiffness that is greater than the other portion of the distal body end portion 208 to which it is joined.

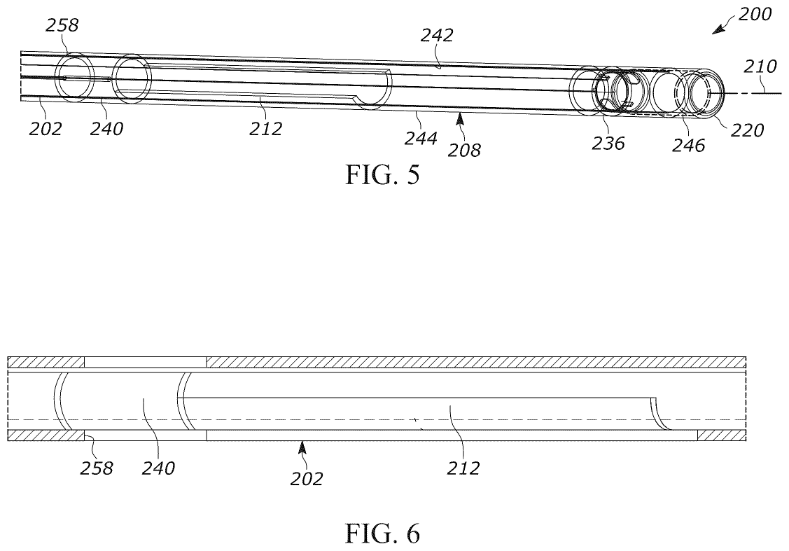

FIG. 5 is another view of the sheath of FIG. 3 showing features within the sheath 200 that are hidden in the view of FIG. 3. FIG. 6 is an enlarged view of an intermediate part of the sheath of FIG. 5. For example, in FIG. 5 the tubular body 202 has interior and exterior surfaces 242 and 244, respectively, spaced apart from each other by one or more layers that define the sidewall of the tubular body. A distal sensor 246 is also shown in FIG. 5 within the tubular body 202. For example, the distal sensor 246 includes one or more position sensors having a known fixed location with respect to the distal end 220. The position sensor can be configured to provide a sensor signal responsive to an electromagnetic (EM) field (e.g., provided by a field generator of an EM navigation system). A position of the sensor 246 can be determined in a spatial coordinate system (e.g., a 3D navigation coordinate system) based on a sensor signal provided by the position sensors that represents the sensor's position in a spatial domain of a navigation system. One or more conductive wires (e.g., a twisted pair) can be coupled to the sensor 246 to carry the sensor signal from the sensor 246. The conductive wires could be insulated. For example, the wires can pass through a respective sensor lumen 262, formed within the sidewall of the tubular body 202 or along an interior of the interior surface thereof. Also, or as an alternative, one or more other types of sensors (e.g., a force sensor, temperature sensor, electrical sensor, etc.) can be implemented at or near the distal end 220 to provide multi-modal sensing functionality.

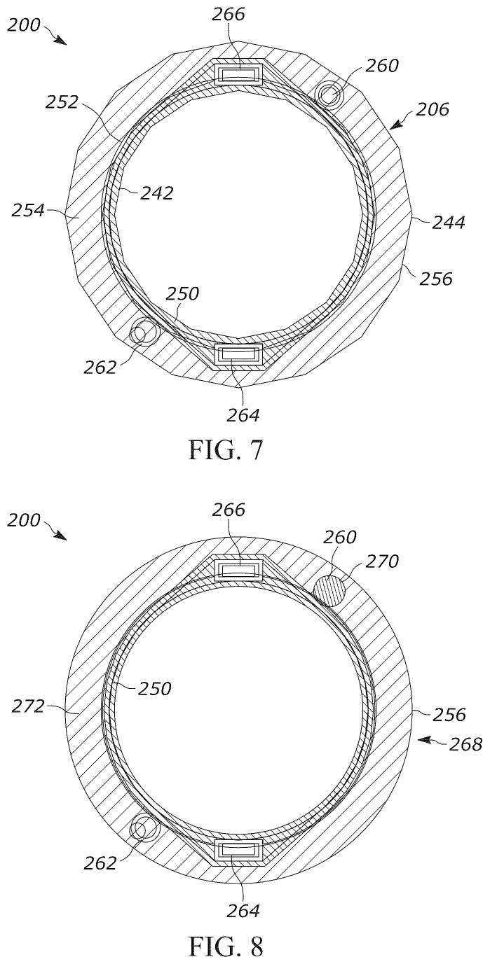

FIG. 7 is a cross-sectional view taken along line 7-7 of FIG. 2 through the proximal body portion 206. The sidewall 202 of the proximal body portion 206 includes multiple layers (from the interior surface 242 outward), including a liner layer (e.g., PTFE or other polymer) 250, a braided section (e.g., metal braids) 252, a polymer layer 254 and an outer jacket layer 256. Each such layer can have a cylindrical shape that extends the length of the proximal body portion 206 from an axial location at or near the ports to a distal end 258 of the proximal body portion. In some examples, the liner 250 and braided layer 252 extend longitudinally through approximately the entire length of the sidewall of the sheath 200, and different polymer layers can be provided at different axial segments, such as described herein.

The sidewall 202 of the proximal body portion 206 also includes one or more lumens 260, 262, 264, and 266. Each of the lumens 260, 262, 264, and 266 can include respective liners of a polymer (e.g., PTFE) or other material. For example, the lumens 260 and 262 can have circular cross-sectional shapes dimensioned and configured to receive one or more sensor wires therein, which can be coupled to respective sensors implemented in the sheath 200. The lumen 260 provides a passage for a sensor wire that is coupled to a proximal sensor 270 (see, e.g., FIG. 8) and the lumen 262 provides for passage of a sensor wire coupled to the distal sensor 246. The other lumens 264 and 266 can be dimensioned and configured to provide for axial movement of respective steering cables 232 and 234 therein. For example, the lumens 264 and 266 can have flat or rectangular cross-sectional shapes and the steering cables can also be flat or have rectangular cross-sectional shapes adapted to move axially within the respective lumens during actuation thereof to effect deflection of the distal body portion of the sheath, such as described herein.

FIG. 8 is a cross-sectional view taken along line 8-8 of FIG. 2 at a transition region 268 of the sidewall 202, which is located axially between the transition region 240 and the proximal body portion 206. The transition region 268 extends an axial length and can include (or contain) a sensor 270, which can reside within a polymer layer 272. The jacket 256 can enclose the polymer layer. In an example, the sensor 270 is a position sensor having a known fixed location and orientation on the sheath, which location and orientation are also known relative to the position and orientation of the distal position sensor 246. The position sensor 270 can include a coil wound around a magnetic core, and is configured to provide a sensor signal responsive to an EM field. In one example, the position sensor 270 is an electromagnetic sensor (e.g., a sensor coil) configured to sense a plurality of degrees of freedom (DOF) in response to an electromagnetic field, such as provided by a field generator of the Aurora electromagnetic tracking system commercially available from Northern Digital Inc. of Waterloo, Ontario, Canada. In an example, the position sensor 270 is a 5 or 6 DOF tracking sensor, including an electrically conductive coil, which provides an electrical signal (e.g., current) responsive to an electromagnetic field from a field generator. A position of the sensor 270 thus can be determined in a navigation coordinate system based on a sensor signal provided by the position sensor. Other types of sensors can be used as the sensor 270. One or more wires (e.g., a twisted pair) can be coupled to the sensor 270 and pass through the lumen 260 to carry the sensor signal from the sensor to a proximal connector (e.g., on or extending from a handle of the sheath).

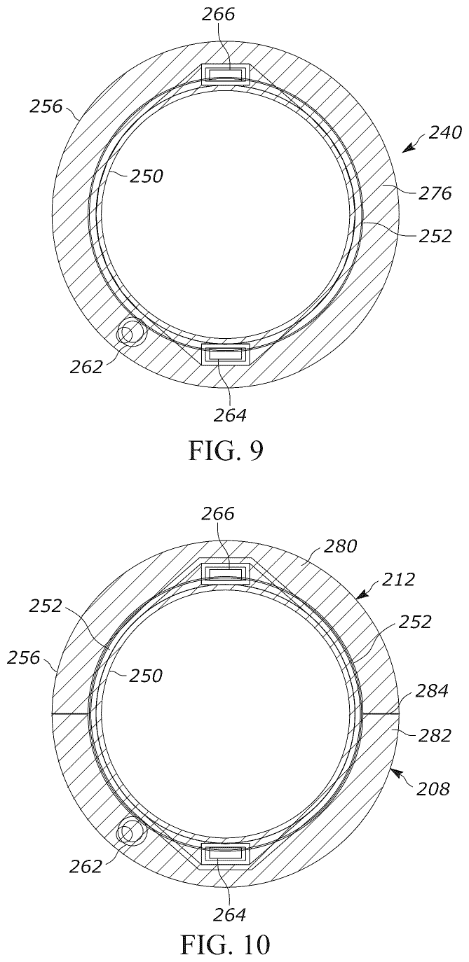

FIG. 9 is a cross-sectional view taken along line 9-9 of FIG. 2 through the transition region 240 of the sidewall 202, which is located between the transition region 268 and the distal body end portion 208. In other contexts one or both of the transition regions 240 and 268 can be considered part of the distal end body portion 208. In an example, the sidewall 272 of proximal transition region 268 has a stiffness that is greater than the stiffness of a polymer sidewall 276 of the distal transition region 240. In other examples, the stiffness can decrease axially distally through the transition regions by other amounts, which can be stepped or continuous amounts.

FIG. 10 is a cross-sectional view taken along line 10-10 of FIG. 2 through the deflection control portion 212. In the example of FIG. 10, the deflection control portion 212 includes a polymer layer 280 having a semi-cylindrical cross-sectional shape that is disposed over the braided layer 252. A diametrically opposed portion of the distal body end portion 208 includes a polymer layer 282 disposed over the opposing portion of the braided layer, and the braided layer 252 is over the cylindrical inner liner layer 250. As shown, the longitudinally extending edges of the polymer layers 280 and 282 are joined together, shown at 284, around the braided layer 252 (or around another intermediate layer not shown). For example, the edges of the polymer layer 280 of the deflection control portion are fused to adjacent edges of other polymer sidewall portions by thermal reflow or by another joining method (e.g., ultrasonic welding, adhesives, etc.). As described herein, the deflection control portion 212 has a different stiffness than a diametrically opposed portion of the distal body end portion 208. For example, the polymer layer 280 of the deflection control portion 212 has a stiffness that is greater than the stiffness of the polymer layer 282.

As a further example, the deflection control portion 212 is formed of a polymer construction having a stiffer durometer to limit bend diameter in the direction of the deflection. Examples of materials that can be used to implement deflection control portion 212 stiffer durometer polymers, such as various grades of Nylons 11 & 12 (e.g., VESTAMID® polyamide resins, GRILAMID® polyamides, RISLAMID® polyamides, and the like). These and other materials suitable for the deflection control portion 212, for example, have a durometer greater than 72 Shore D. The sidewall portion of the distal end portion in the deflection zone opposite the deflection control portion 212 is more compliant (e.g., easier to deflect) than the deflection control portion 212, and can be constructed from softer durometer materials (e.g., having a durometer less than 60 Shore D). Examples of materials that can be used to form the sidewall portion of the distal end portion 208 in the deflection zone opposite the deflection control portion 212 include thermoplastic polyurethane, such as in the Shore A durometer scale. Other example materials that can be used to form the sidewall portion of the distal end portion 208 in the deflection zone opposite the deflection control portion 212 include PEBAX polymers or ARNITEL thermoplastic copolyester materials (e.g., having a durometer less than 60 Shore D, such as in the range from about 25 to about 45 Shore D). Other materials having suitable material and biocompatible properties can be used in other examples.

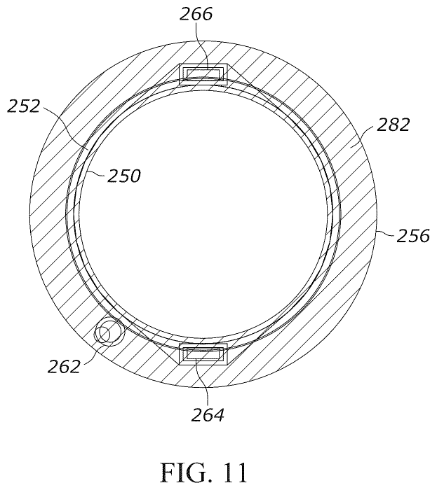

FIG. 11 is a cross-sectional view taken along line 11-11 of FIG. 2 of the distal body end portion 208 at location between the deflection control portion 212 and the distal end 220. The cross-sectional view includes a polymer layer, which can be the same as polymer layer 282 having a continuous circular cross-sectional shape of the same polymer material.

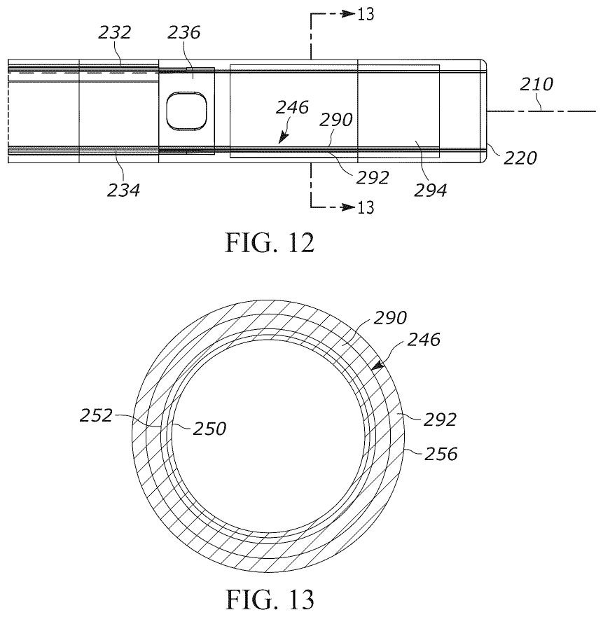

FIG. 12 is an enlarged view of a distal end part of the sheath of FIG. 5. FIG. 12 includes the anchor ring 236 from which the steering cables 232 and 234 extend through respective lumens 266 and 264. FIG. 12 also illustrates the distal sensor 246 as including a cylindrical core body 290, which can include a number of conductive windings around the core body. The anchor ring 236 and distal sensor 290 can be mounted (partially or wholly) in an outer over ring 292 to increase stiffness and deflection of the distal body end portion responsive to actuation of the respective steering cables 232 and 234. A flexible distal tip 294 can extend from the outer over ring 292 and core body of the sensor 246 to define the open distal end 220 of the sheath 200.

FIG. 13 is a cross-sectional view taken along line 13-13 of FIG. 12 through the distal sensor 236. As shown in FIGS. 12 and 13, the core body and windings 290 of the sensor 246 can be disposed around the braided layer 252 and inner liner 250.

While in the example of FIGS. 1-14, the deflection control portion (e.g., a cylindrical sector segment) 112, 212 is described at a fixed position within the distal body end portion 108, 208 spaced from the distal end 120, 220, in other examples, the deflection control portion 112, 212 can include a stiffening element having a distal portion that is axially moveable within a cylindrical sector of distal body end portion 108, 208. In such an example, the length of the stiffening element within the distal body end portion 108, 208 and the material properties of the stiffening element (and other parts of the distal body end portion) define first and second bend angles thereof.

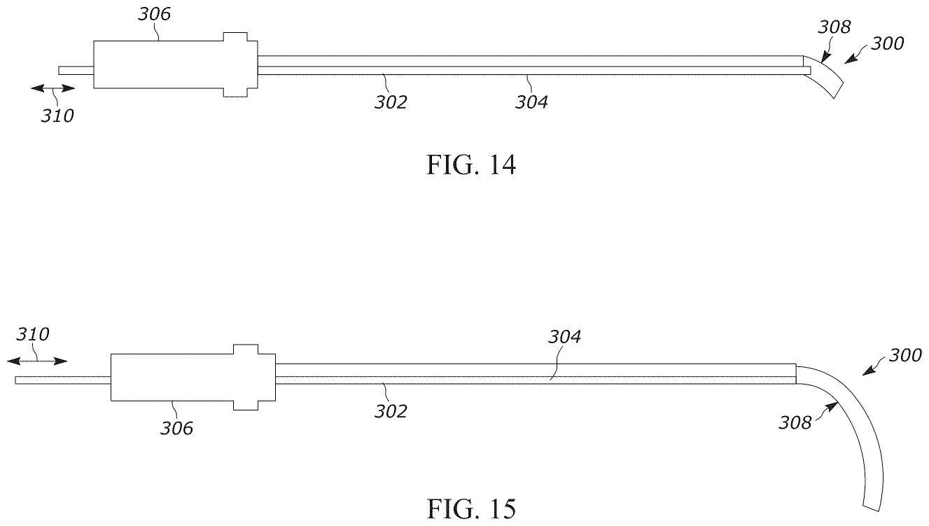

By way of example, FIGS. 14 and 15 depict a sheath 300 having variable bend diameters which can be controlled as a function of a stiffening element 302 that is axially moveable within or along a sidewall of the sheath. For example, the stiffening element 302 can be in the form of an elongated flexible rod (e.g., having a circular, rectangular or other cross-sectional shape), which is moveable within a respective stiffening lumen 304 extending axially within or along a tubular sidewall of the sheath. Similar to the examples of FIGS. 1-13, the stiffening lumen 304 and the stiffening element 302 reside on a given half of a cross-section of the sheath, such as aligned substantially radially (inwardly or outwardly) from a given steering lumen that provides for the passage of a respective steering cable. In other examples, the stiffening lumen 304 can be angularly offset from the given steering lumen. The stiffening element 302 can be moved axially through the stiffening lumen (in directions shown by arrow 310) to an axial position relative to the distal body end portion 308 to adjust the bend angle of a deflection zone of the distal body end portion of the sheath 300. That is, the bend angle of the distal body end portion 308 during steering is variable depends on the axial position of the stiffening element 302 within the stiffening lumen. In each of the examples of FIGS. 14 and 15, the axial position of the stiffening element 302 defines two different bend angles for the distal body end portion depending on its given position within the distal body end portion 308.

In an example, a user can adjust the axial position of the stiffening element 302 by pushing or pulling on a proximal end of the stiffening element 302 extending from a handle 306, which also includes one or more steering manipulators. In another example, the handle could include an additional manipulator to adjust the axial position of the stiffening element to define the bend angles (e.g., radius of curvature) for the distal body endo portion. The sheath 300 can also include a locking mechanism (e.g., integrated in the handle) adapted to hold the stiffening element 302 at a given fixed position to set first and second bend angles in opposite directions along a plane extending through the stiffening element and a longitudinal axis of the distal body end portion. In other examples, such as in a unidirectional steerable sheath, the bend angle in one of the directions can be varied according to the given fixed position of the stiffening element during steering.

As described herein, a sheath 100, 200, 300 is configured to provide multiple different bend angles. The sheath 100, 200, 300 can include any number and arrangement of sensors for a particular use environment, including localization sensors for detecting the position of such sensors by a navigation system. An example navigation system and use environment is disclosed in U.S. Pat. No. 10,799,145, which is incorporated herein by reference. For example, the navigation system includes a tracking system that includes a field generator to provide an EM field. The tracking system is coupled to the tracking sensor 246 and 270 through a communications link (e.g., physical or wireless link). In an example, the tracking system thus is operative to receive electrical signals (e.g., current) from the tracking sensors 246 and 270 responsive to the EM field produced by the field generator. In another example, the tracking system may provide a signal to the tracking sensor through the communication link that generates a field that is sensed by respective sensors of the tracking system. The tracking system is configured to determine a position and orientation of the tracking sensor in a three-dimensional coordinate system of the tracking system responsive to the electrical signal from the tracking sensor. In other examples, different types of signal processing and analysis from other types of sensors implemented on the sheath 100, 200, 300 may be implemented by external electronics and computing systems.

In view of the foregoing, a sheath 100, 200, 300, as described herein, allows a user (e.g., surgeon) to access to more vessels with a single sheath device because the sheath can be steered along more than one curvature for accessing different vessels within the body. Depending on the vessel, for example, it may be easier to access it using a smaller curvature or a larger curvature, which are provided on the sheath 100, 200, 300.

In other examples, the sheath 100, 200, 300 described herein can also be configured as a catheter, cannula, surgical instrument, endoscope, or introducer. Such devices are used to deliver tools, implants, or other items to the body. The variable curvature (e.g., bend angle) of the sheath (or catheter or other device) described herein thus will allow the surgeon better alignment for using these tools or delivering the implants or other items to the body. Such variable diameter sheaths and catheters will reduce procedure time by allowing the surgeon to use one sheath or catheter instead of multiple sheaths or catheters to do the same task. Additionally, these sheaths and catheters are expected to reduce adverse events by affording users of the sheath better control.

From the perspective of a device supplier or institution (e.g., hospital or surgical center) that stocks such items for user by its physicians, the sheath 100, 200, 300 described herein enables multiple single diameter sheath units to be consolidated into a single sheath unit 100, 200, 300 having at least two different bend diameters. This is in contrast to many existing sheath designs in which each sheath can accommodate only a single bend diameter.

The sheath 100, 200, 300 further can include any desired seals, gaskets, connectors, and/or other components provided to the sheath as appropriate for a particular use environment, and can readily be provided by one of ordinary skill in the art taking into account, for example, durability, affordability, sterilizability, case of manufacture, and/or any other desired factors or combinations thereof.

### EXAMPLE EMBODIMENTS

Several aspects of the present technology are set forth in the following numbered examples.

Example 1. A sheath comprising:
an elongate tubular body having a cylindrical sidewall defining a lumen and having longitudinally spaced apart proximal and distal body end portions, the distal body end portion including a first sector segment extending longitudinally along the distal body end portion between first and second ends thereof, in which the first sector segment is adapted to provide the sidewall of the distal body end portion along the first sector segment a different stiffness than a diametrically opposed portion of the sidewall of the distal body end portion, and the distal body end portion is adapted for bidirectional deflection thereof with different amounts of curvature in opposing first and second directions along a virtual plane extending through the first sector segment and a central axis of the distal body end portion.

Example 2. The sheath of example 1, wherein the different amounts of curvature in opposing first and second directions depends on a relative stiffness between the first sector segment and at least the diametrically opposed portion of the sidewall of the distal body end portion.

Example 3. The sheath of example 2, wherein the stiffness of the first sector segment is greater than a stiffness of the at least the diametrically opposed portion of the sidewall of the distal body end portion, and the distal body end portion is adapted to deflect with a smaller bend diameter in the first direction around the first sector segment than in the second direction away from the first sector segment.

Example 4. The sheath according to any one of examples 1, 2 or 3, wherein the distal body end portion extends a length from the proximal body end portion between proximal and distal ends thereof, and the first sector segment has an axial length that is less than the length of the distal body end portion.

Example 5. The sheath of example 4, wherein the first sector segment has proximal and distal ends spaced apart from each other by edges thereof that extend an axial length longitudinally along the sidewall of the distal body end portion, and the edges of the first sector segment are spaced apart from each other to define an arc length of the first sector segment.

Example 6. The sheath of example 5, wherein the edges and at least the distal end of the first sector segment abut and are joined with the sidewall of the distal body end portion, and the proximal end of the first sector segment abuts and is joined with the sidewall of either the distal body end portion or the proximal body end portion.

Example 7. The sheath according to any one of examples 5 or 6, wherein a portion of the distal body end portion extends distally from the distal end of the of the first sector segment to terminate in an open end thereof.

Example 8. The sheath according to any preceding example, further comprising:
an anchor coupled to the sidewall of the distal body end portion at a location between a distal end of the first sector segment and the distal end of the distal body end portion; and
first and second steering cables coupled to the anchor to provide for the bidirectional deflection of the distal body end portion in the first and second directions.

Example 9. The sheath according to any preceding example, further comprising at least one sensor mounted at a known location of the distal body end portion.

Example 10. The sheath of example 9, wherein the at least one sensor comprises:
a distal navigation sensor mounted to the distal body end portion at an axial location between the distal end and the first sector segment.

Example 11. The sheath according to any preceding example, wherein the sidewall of the distal body end portion has a circular cylindrical cross-sectional shape, and the first sector segment has a cross-sectional arc length that is less than or equal to about one-half a circumference of the sidewall of the distal body end portion.

Example 12. The sheath of example 11, wherein the first sector segment has a semi-cylindrical cross-sectional shape.

Example 13. An elongate sheath includes a deflection varying portion extending longitudinally within a sidewall of a deflectable distal body end portion, in which the deflection varying portion has a relative stiffness that differs from at least a diametrically opposed axially portion of the sidewall of the distal body end portion, a portion of the distal body end portion extends distally from the deflection varying portion, whereby the distal body end portion has different bend diameters for deflection along a plane in a first direction towards the deflection varying portion and/or in a second direction away from the deflection varying portion depending on the relative stiffness.

Example 14. The sheath of example 13, wherein the stiffness of the deflection varying portion is greater than a stiffness of the at least the diametrically opposed portion of the sidewall of the distal body end portion, and the distal body end portion is configured to deflect with a smaller bend diameter in a direction towards the deflection varying portion than in a direction away from the deflection varying portion.

Example 15. The sheath according to any of examples 13 or 14, wherein the distal body end portion extends from a proximal body end portion between proximal and distal ends thereof to define an axial length of the distal body end portion, and the deflection varying portion has an axial length that is less than the axial length of the distal body end portion.

Example 16. The sheath of example 15, wherein the deflection varying portion defines a cylindrical sector segment having proximal and distal ends spaced apart from each other by edges thereof that extend an axial length longitudinally along the sidewall of the distal body end portion, and the edges of the cylindrical sector segment are spaced apart from each other to define an arc length of the cylindrical sector segment.

Example 17. The sheath of example 16, wherein the edges and at least the distal end of the cylindrical sector segment abut and are joined with the sidewall of the distal body end portion, and the proximal end of the cylindrical sector segment abuts and is joined with the sidewall of either the distal body end portion or the proximal body end portion.

Example 18. The sheath according to any one of examples 13 or 14, further comprising a stiffening lumen extending axially within the distal body end portion, in which the deflection varying portion includes an elongated stiffening element that is axially moveable within the stiffening lumen to define the relative stiffness of the deflection varying portion based on an amount of stiffening element extending axially within the stiffening lumen.

Example 19. The sheath according to any one of examples 13, 14, 15, 16, 17, or 18, further comprising:
an anchor coupled to the sidewall of the distal body end portion at a location between a distal end of the deflection varying portion and the distal end of the distal body end portion; and
at least one steering cable coupled to the anchor to provide for deflection of the distal body end portion in at least one of the first and second directions.

Example 20. A sheath comprising:
an elongate tubular body having a cylindrical sidewall defining a lumen and having longitudinally spaced apart proximal and distal body end portions, the distal body end portion including a first semi-cylindrical sector segment defining a deflection control region extending longitudinally along the distal body end portion between first and second ends thereof, in which the deflection control region has a different stiffness relative to a diametrically opposed region of the distal body end portion, such that the distal body end portion is configured to provide for bidirectional deflection thereof with different amounts of curvature in opposing first and second directions along a virtual plane extending through the deflection control region, the diametrically opposed region of the distal body end portion, and a central axis of the distal body end portion.

As used herein, the term “and/or” can include any and all combinations of one or more of the associated listed items.

As used herein, phrases and/or drawing labels such as “X-Y”, “between X and Y” and “between about X and Y” can be interpreted to include X and Y.

It will be understood that when an element is referred to as being “on,” “attached” to, “connected” to, “coupled” with, “contacting”, “adjacent”, etc., another element, it can be directly on, attached to, connected to, coupled with, contacting, or adjacent the other element or one or more intervening elements may also be present. In contrast, when an element is referred to as being, for example, “directly on,” “directly attached” to, “directly connected” to, “directly coupled” with, “directly contacting”, or “directly adjacent” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature might not have portions that overlap or underlie the adjacent feature.

Spatially relative terms, such as “under,” “below,” “lower,” “over,” “upper”, “proximal”, “distal”, and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the figures.

Additionally, any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment.

The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

1. A sheath comprising:
an elongate tubular body having a cylindrical sidewall defining a lumen and having longitudinally spaced apart proximal and distal body end portions, the distal body end portion including a first sector segment extending longitudinally along the distal body end portion between first and second ends thereof, in which the first sector segment is adapted to provide the sidewall of the distal body end portion along the first sector segment a different stiffness than a diametrically opposed portion of the sidewall of the distal body end portion, and the distal body end portion is adapted for bidirectional deflection thereof with different amounts of curvature in opposing first and second directions along a virtual plane extending through the first sector segment and a central axis of the distal body end portion.

2. The sheath of claim 1, wherein the different amounts of curvature in opposing first and second directions depends on a relative stiffness between the first sector segment and at least the diametrically opposed portion of the sidewall of the distal body end portion.

3. The sheath of claim 2, wherein the stiffness of the first sector segment is greater than a stiffness of the at least the diametrically opposed portion of the sidewall of the distal body end portion, and the distal body end portion is adapted to deflect with a smaller bend diameter in the first direction around the first sector segment than in the second direction away from the first sector segment.

4. The sheath of claim 1, wherein the distal body end portion extends a length from the proximal body end portion between proximal and distal ends thereof, and the first sector segment has an axial length that is less than the length of the distal body end portion.

5. The sheath of claim 4, wherein the first sector segment has proximal and distal ends spaced apart from each other by edges thereof that extend an axial length longitudinally along the sidewall of the distal body end portion, and the edges of the first sector segment are spaced apart from each other to define an arc length of the first sector segment.

6. The sheath of claim 5, wherein the edges and at least the distal end of the first sector segment abut and are joined with the sidewall of the distal body end portion, and the proximal end of the first sector segment abuts and is joined with the sidewall of either the distal body end portion or the proximal body end portion.

7. The sheath of claim 5, wherein a portion of the distal body end portion extends distally from the distal end of the of the first sector segment to terminate in an open end thereof.

8. The sheath of claim 1, further comprising:
an anchor coupled to the sidewall of the distal body end portion at a location between a distal end of the first sector segment and the distal end of the distal body end portion; and
first and second steering cables coupled to the anchor to provide for the bidirectional deflection of the distal body end portion in the first and second directions.

9. The sheath of claim 1, further comprising at least one sensor mounted at a known location of the distal body end portion.

10. The sheath of claim 9, wherein the at least one sensor comprises:
a distal navigation sensor mounted to the distal body end portion at an axial location between the distal end and the first sector segment.

11. The sheath of claim 1, wherein the sidewall of the distal body end portion has a circular cylindrical cross-sectional shape, and the first sector segment has a cross-sectional arc length that is less than or equal to about one-half a circumference of the sidewall of the distal body end portion.

12. The sheath of claim 11, wherein the first sector segment has a semi-cylindrical cross-sectional shape.

13. An elongate sheath includes a deflection varying portion extending longitudinally within a sidewall of a deflectable distal body end portion, in which the deflection varying portion has a relative stiffness that differs from at least a diametrically opposed axially portion of the sidewall of the distal body end portion, a portion of the distal body end portion extends distally from the deflection varying portion, whereby the distal body end portion has different bend diameters for deflection along a plane in a first direction towards the deflection varying portion and/or in a second direction away from the deflection varying portion depending on the relative stiffness.

14. The sheath of claim 13, wherein the stiffness of the deflection varying portion is greater than a stiffness of the at least the diametrically opposed portion of the sidewall of the distal body end portion, and the distal body end portion is configured to deflect with a smaller bend diameter in a direction towards the deflection varying portion than in a direction away from the deflection varying portion.

15. The sheath of claim 13, wherein the distal body end portion extends from a proximal body end portion between proximal and distal ends thereof to define an axial length of the distal body end portion, and the deflection varying portion has an axial length that is less than the axial length of the distal body end portion.

16. The sheath of claim 15, wherein the deflection varying portion defines a cylindrical sector segment having proximal and distal ends spaced apart from each other by edges thereof that extend an axial length longitudinally along the sidewall of the distal body end portion, and the edges of the cylindrical sector segment are spaced apart from each other to define an arc length of the cylindrical sector segment.

17. The sheath of claim 16, wherein the edges and at least the distal end of the cylindrical sector segment abut and are joined with the sidewall of the distal body end portion, and the proximal end of the cylindrical sector segment abuts and is joined with the sidewall of either the distal body end portion or the proximal body end portion.

18. The sheath of claim 13, further comprising a stiffening lumen extending axially within the distal body end portion, in which the deflection varying portion includes an elongated stiffening element that is axially moveable within the stiffening lumen to define the relative stiffness of the deflection varying portion based on an amount of stiffening element extending axially within the stiffening lumen.

19. The sheath of claim 13, further comprising:
an anchor coupled to the sidewall of the distal body end portion at a location between a distal end of the deflection varying portion and the distal end of the distal body end portion; and
at least one steering cable coupled to the anchor to provide for deflection of the distal body end portion in at least one of the first and second directions.

20. A sheath comprising:
an elongate tubular body having a cylindrical sidewall defining a lumen and having longitudinally spaced apart proximal and distal body end portions, the distal body end portion including a first semi-cylindrical sector segment defining a deflection control region extending longitudinally along the distal body end portion between first and second ends thereof, in which the deflection control region has a different stiffness relative to a diametrically opposed region of the distal body end portion, whereby the distal body end portion is configured to provide for bidirectional deflection thereof with different amounts of curvature in opposing first and second directions along a virtual plane extending through the deflection control region, the diametrically opposed region of the distal body end portion, and a central axis of the distal body end portion.
